(19) United States
(12) Patent Application Publication
(10) Pub. No.: US 2017/0202512 A1
(43) Pub. Date: Jul. 20, 2017

(54) ELECTROCARDIOGRAPHY SCANNER MODULE, MULTI-CONTACT CONNECTOR THEREOF, ELECTROCARDIOGRAPHY SCANNER THEREOF AND SMART CLOTHES USING THE SAME

(71) Applicants: LITE-ON ELECTRONICS (GUANGZHOU) LIMITED, Guangzhou (CN); LITE-ON TECHNOLOGY CORPORATION, Taipei (TW)

(72) Inventor: Chuan-Feng WU, Taipei (TW)

(73) Assignees: LITE-ON ELECTRONICS (GUANGZHOU) LIMITED, Guangzhou (CN); LITE-ON TECHNOLOGY CORPORATION, Taipei (TW)

(21) Appl. No.: 15/231,265

(22) Filed: Aug. 8, 2016

Related U.S. Application Data
(60) Provisional application No. 62/278,996, filed on Jan. 15, 2016.

(30) Foreign Application Priority Data
Apr. 14, 2016 (CN) 201610235716.9

(51) Int. Cl. A61B 5/00; A61B 5/0408; A61B 5/145; A61B 5/021; A61B 5/024; H01R 24/00; A61B 5/0285

(52) U.S. Cl. CPC A61B 5/6804; H01R 24/005; A61B 5/0408; A61B 5/0285; A61B 5/021; A61B 5/02438; A61B 5/4872; A61B 5/14532; A61B 5/14542; H01R 2201/12

(57) ABSTRACT

An electrocardiography (ECG) scanner module, a multi-contact connector thereof, an ECG scanner thereof and smart clothes using the same are provided. The multi-contact connector includes a base, a fitting portion and several first conductive portions. The base is disposed on a wearable carrier having several conductive wires. The fitting portion is disposed on the base and surrounds a receiving portion. The first conductive portions are annularly disposed on an inner periphery surface of the receiving portion, and each first conductive portion connects to the corresponding conductive wire. The ECG scanner includes several second conductive portions, and an end of each second conductive portion is projected from an outer periphery surface of the ECG scanner. When the multi-contact connector connects to the ECG scanner, each second conductive portion provides a radial force for connecting to the corresponding first conductive portion.

This application claims the benefit of U.S. provisional application Ser. No. 62/278,996, filed Jan. 15, 2016, the subject matter of which is incorporated herein by reference, and claims the benefit of People's Republic of China application Serial No. 201610235716.9, filed Apr. 14, 2016, the subject matter of which is incorporated herein by reference.

### BACKGROUND

Technical Field

The disclosure relates in general to an electrocardiography (ECG) scanner module, a connector thereof, an ECG scanner thereof and smart clothes using the same, and more particularly to an ECG scanner module with multiple contacts, a multi-contact connector thereof, an ECG scanner thereof and smart clothes using the same.

Background

Conventional smart clothes includes a connector embedded in cloth of the smart clothes for obtaining physiological signal of human body. The ECG scanner is configured to be connected to the connector for receiving and processing the physiological signal of human body from the connector. In one connecting method, the ECG scanner includes a male metal buckle such as a male copper buckle, and the connector includes a female metal buckle such as a female copper buckle. The ECG scanner and the connector connect to each other by buckling the male copper buckle into the female copper buckle along an axial direction.

However, such conventional method has a disadvantage: the more the number of the contacts is, the larger the contacting area of the male copper buckles and the female copper buckles is, and accordingly a larger pulling and inserting force is needed. As a result, the comfort of the human body wearing the smart clothes and diversity of the functions will be negatively affected. In addition, since the male copper buckle and the female copper buckle are clearance fit, when user moves, the conventional copper buckles generate unnecessary noise due to unsteady of the connecting impedance resulted from shocking. Furthermore, since a pair of copper buckles only transmits one signal, the ECG scanner becomes larger when the number of copper buckles is required to be more, such as eight. As a result, the smart clothes make the user uncomfortable. Thus, conventional copper buckles are not conducive to the design of multiple signals, such as the measurements of multi-lead ECG signal, body fat, blood glucose, etc.

Thus, it is needed to provide a new connector for solving conventional problems.

### SUMMARY

According to one embodiment of the invention, a multi-contact connector is provided. The multi-contact connector is adapted to be disposed on a wearable carrier and connected to an electrocardiography (ECG) scanner, wherein the wearable carrier has a plurality of conductive wires, the ECG scanner has a plurality of second conductive portions, each of the second conductive portions has a first end projecting from an outer circumferential surface of the ECG scanner, and the multi-contact connector comprises a base, a fitting portion and a plurality of first conductive portions. The base is disposed on the wearable carrier. The fitting portion is disposed on the base and surrounds a first receiving portion. The first conductive portions are annularly disposed on an inner circumferential surface, wherein each first conductive portion is electrically connected to the corresponding conductive wire. Wherein when the ECG scanner and the multi-contact connector connect to each other, each second conductive portion provides a radial force along a radial direction for being electrically connected to the corresponding first conductive portion.

According to another embodiment of the invention, an ECG scanner is provided. The ECG scanner is adapted to be connected to a multi-contact connector having a plurality of first conductive portions. The ECG scanner comprises a main body and a plug component. The plug component is disposed on the main body and comprises a casing and a plurality of second conductive portions. The casing has a circumferential wall. The second conductive portions have deformability and are annularly arranged in the circumferential wall of the casing, wherein a first end of each second conductive portion projects from an outer circumferential surface of the circumferential wall. Wherein when the ECG scanner and the multi-contact connector connect to each other, each second conductive portion provides a radial force along a radial direction for being electrically connected to the corresponding first conductive portion.

According to another embodiment of the invention, an ECG scanner module is provided. The ECG scanner module includes the multi-contact connector as mentioned above and the ECG scanner as mentioned above. The ECG scanner is configured to be connected to the multi-contact connector, wherein each second conductive portion of the ECG scanner is configured to be contacted to the corresponding first conductive portion.

According to another embodiment of the invention, smart clothes is provided. The smart clothes includes the multi-contact connector as mentioned above. Wherein the smart clothes covers a portion of the multi-contact connector, another portion of the multi-contact connector is exposed from the smart clothes, and the first conductive portions of the multi-contact connector are exposed from the smart clothes.

According to another embodiment of the invention, smart clothes is provided. The smart clothes includes the ECG scanner module as mentioned above. wherein the smart clothes covers a portion of the multi-contact connector, another portion of the multi-contact connector is exposed from the smart clothes, the first conductive portions of the multi-contact connector are exposed from the smart clothes, and the ECG scanner is electrically connected to the multi-contact connector by each second conductive portion contacting the corresponding first conductive portion.

The above and other aspects of the invention will become better understood with regard to the following detailed description of the preferred but non-limiting embodiment(s). The following description is made with reference to the accompanying drawings.

### BRIEF DESCRIPTION OF THE DRAWINGS

FIG. 1 shows an exploded view of an ECG scanner module according to an embodiment of the invention;

FIG. 2 shows a diagram of the multi-contact connector being covered by cloth;

FIG. 3 shows a diagram of an opposite side of the multi-contact connector of FIG. 2;

FIG. 4 shows an exploded view of the multi-contact connector of FIG. 1;

FIG. 5 shows a diagram of the smart clothes according to an embodiment of the invention;

FIG. 6 shows an appearance view of the ECG scanner of FIG. 1;

FIG. 7 shows an exploded view of the ECG scanner of FIG. 6;

FIG. 8 shows an appearance view of the plug component of FIG. 7;

FIG. 9 shows an exploded view of the plug component of FIG. 8;

FIG. 10 shows an assembly view of a casing and a plurality of second conductive portions of FIG. 9; and

FIG. 11 shows a connecting process of the ECG scanner (the main body is not shown) of FIG. 1 and the multi-contact connector of FIG. 2.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

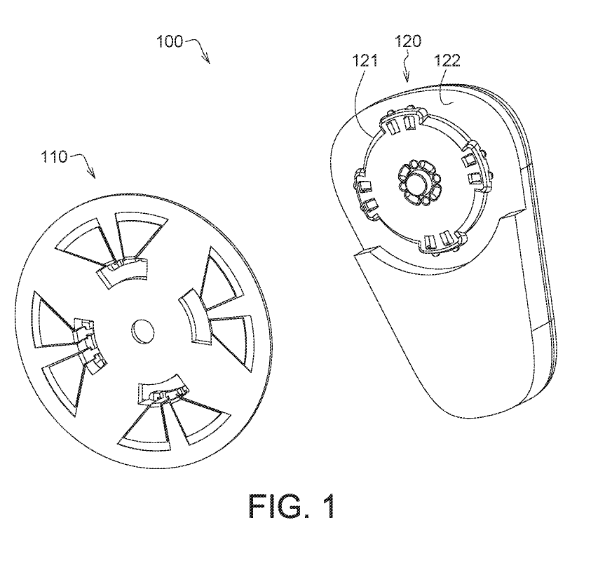

FIG. 1 shows an exploded view of an ECG scanner module 100 according to an embodiment of the invention.

The ECG scanner module 100 of the present embodiment may be combined with smart clothes for the requirement for health and athletics. The ECG scanner module 100 may receive, transmit and analyze physiological signal from human body or animal, such that the management of health and athletics may be much intelligentized.

The ECG scanner module 100 includes a multi-contact connector 110 and an ECG scanner 120. The ECG scanner 120 includes a plug component 121 and a main body 122, wherein the plug component 121 may insert and is electrically connected to the main body 122.

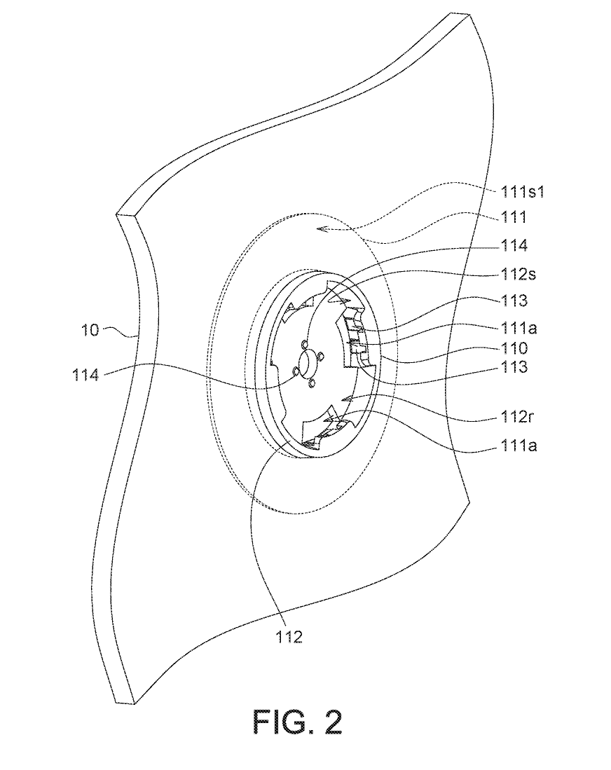

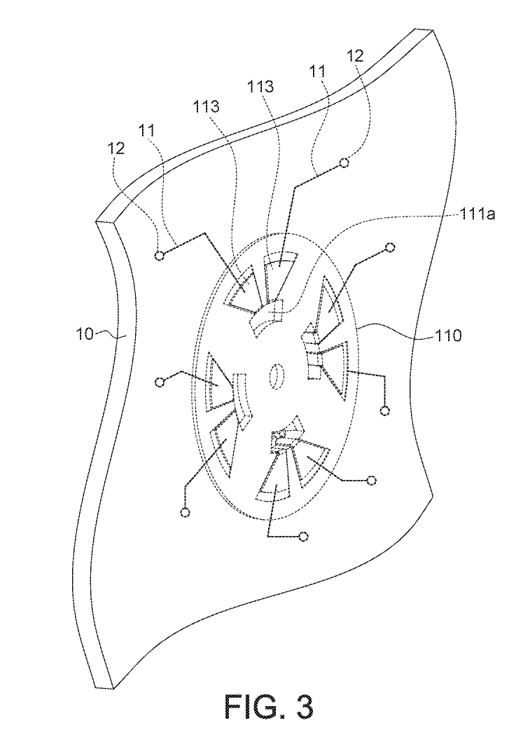

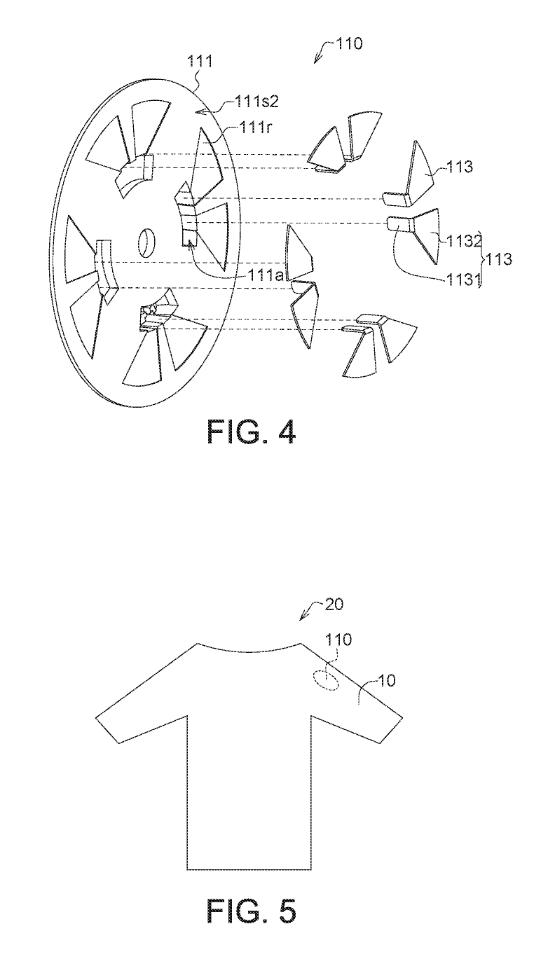

Referring to FIGS. 2-4, FIG. 2 shows a diagram of the multi-contact connector 110 being covered by cloth, FIG. 3 shows a diagram of an opposite side of the multi-contact connector 110 of FIG. 2, and FIG. 4 shows an exploded view of the multi-contact connector 110 of FIG. 1.

The base 110 includes a base 111, a fitting portion and a plurality of first conductive portions 113, wherein the fitting portion is, for example, a flange 112. The flange 112 is disposed on the base 111 and projects from a first surface 111s1 of the base 111. The flange 112 surrounds a first receiving portion 112r. These first conductive portions 113 are annularly and circumferentially disposed on an inner circumferential surface 112s of the first receiving portion 112r.

The base 111 has a plurality of first openings 111a, wherein the first openings 111a pass through the base 111 and interconnect with the first receiving portion 112r. The first conductive portions 113 pass through the first openings 111a and are exposed from the first receiving portion 112r (as shown in FIG. 2). As a result, when the ECG scanner 120 and the multi-contact connector 110 connect to each other, the plug component 121 inserting the first receiving portion 112r may be electrically connected to the first conductive portions 113 of the multi-contact connector 110 exposed from the cloth 10 of the smart clothes.

As shown in FIG. 4, each first conductive portion 113 includes a first sub-conductive portion 1131 and a second sub-conductive portion 1132 connecting to the first sub-conductive portion 1131. Each first sub-conductive portion 1131 passes through the corresponding first opening 111a and is exposed from the first receiving portion 112r. The second sub-conductive portions 1132 are disposed on a second surface 111s2 of the base 111. The base 111 further includes a plurality of recesses 111r extending toward the first surface 111s1 from the second surface 111s2, but not penetrating the base 111. Each recess 111r may receive the corresponding second sub-conductive portion 1132, such that the second sub-conductive portions 1132 are engaged with the base 111, and it may avoid a relative displacement between the base 111 and the second sub-conductive portions 1132. In another embodiment, the second sub-conductive portions 1132 may be adhered to the recesses 111r by adhesion. Alternatively, each sub-conductive portion 1132 may has a size slightly larger than that of the corresponding recess 111r, such that each sub-conductive portion 1132 and the corresponding recess 111r are in tight fit or transition fit. In addition, as shown in FIG. 4, the first sub-conductive portions 1131 and the second sub-conductive portions 1132 are shaped as flat-plate shape or string shape.

In the present embodiment, the number of the first conductive portions 113 is eight. In another embodiment, the number of the first conductive portions 113 may be less or more than eight. The base 111 of the present embodiment has four first openings 111a, wherein each first opening 111a is corresponding to two first conductive portions 113, that is, each first opening 111a allow two first conductive portions 113 to pass through. The number of the first openings 111a and the number of the first conductive portions 113 allowed to pass through the first openings 111a are not limited to the present embodiment. The number of the first opening 111a may depend on mold design, manufacturability, whether the multi-contact connector 110 is easy to be connected to the ECG scanner 120 or the stability of connecting.

As shown in FIG. 3, the cloth 10 is, for example, the cloth of the smart clothes. The cloth 10 includes a plurality of conductive wires 11 and a plurality of pads 12, wherein each conductive wire 11 is electrically connected to the corresponding pad 12. The cloth 10 may be made of fibers, woven, non-woven fabric or any other kind of material which may make smart clothes. In addition, the cloth 10 may further includes at least one conductive layer, at least one plating layer, at least one conductive coating layer and/or the at least one conductive adhesive layer, so as to form the conductive wires 11 of the cloth 10.

One end of each conductive wire 11 is connected to the corresponding pad 12, and another end of each conductive wire 11 is connected to the corresponding first conductive portions 113 of the multi-contact connector 110. When the human body or animal wear the cloth 10, the pads 12 contact a portion of human body or animal for detecting physiological signal of human body or animal. Then, the physiological signal of human body or animal may be transmitted to the first conductive portions 113 of the multi-contact connector 110 through the conductive wires 11.

The multi-contact connector 110 is electrically connected to the conductive wires 11 of the cloth 10 through the first conductive portions 113 for transmitting the physiological signal (current or voltage) of human body or animal detected from the human body or animal to the ECG scanner 120 electrically connected to the multi-contact connector 110, wherein the physiological signal may be transmitted to the main body 122 through the plug component 121. The main body 122 may analyze the physiological signals to obtain physiological information, such as pulse wave velocity (PWV), blood pressure, heart rate, body fat, blood glucose, pulse oximetry (SpO2), etc.

FIG. 5 shows a diagram of the smart clothes 20 according to an embodiment of the invention. The smart clothes 20 includes the multi-contact connector 110 and the cloth 10, whether the multi-contact connector 110 is disposed on the cloth 10. In another embodiment, the smart clothes 20 further includes the ECG scanner 120 which may be connected to the multi-contact connector 110. The multi-contact connector 110, the cloth 10 and the CEG scanner 20 have the features similar to the aforementioned descriptions, and the similarities are not repeated here.

As shown in FIG. 2, the multi-contact connector 110 may be covered by the cloth 10, whether the flange 112 is exposed from or projects from the cloth 10, such that the plug component 121 (as shown in FIG. 1) of the ECG scanner 120 may be connected to the first conductive portions 113 through the exposed or projecting flange 112.

The plug component 121 may be connected to the multi-contact connector 110 by way of inserting or rotating, such that the plug component 121 is electrically connected to and locked to the multi-contact connector 110.

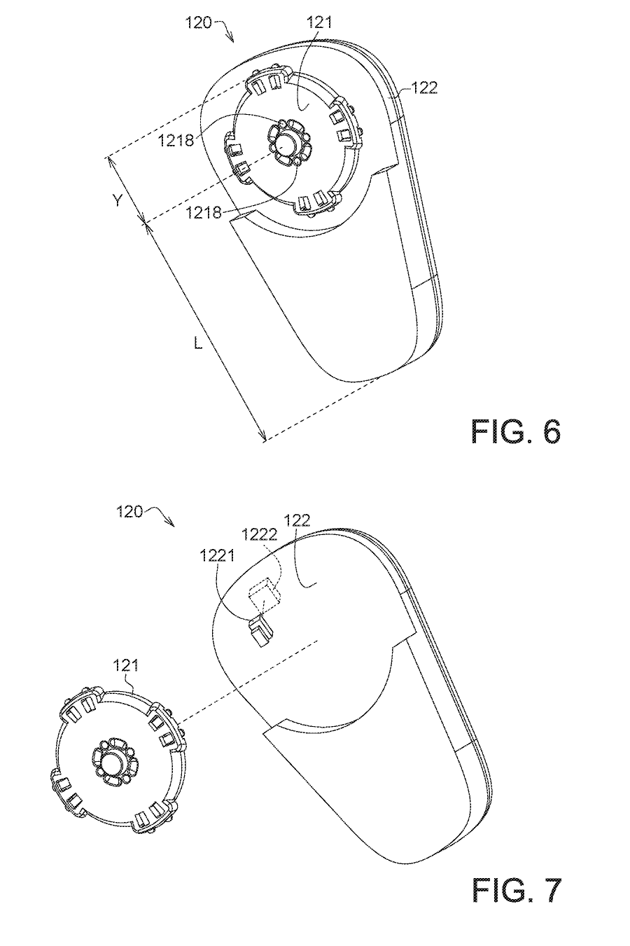

Referring to FIGS. 6 and 7, FIG. 6 shows an appearance view of the ECG scanner 120 of FIG. 1, and FIG. 7 shows an exploded view of the ECG scanner 120 of FIG. 6.

The plug component 121 is disposed on and electrically connected to the main body 122, and accordingly the main body 122 may analyze physiological signals from the plug component 121 to obtain physiological information. For example, as shown in FIG. 7, the ECG scanner 120 further includes a connector 1221 and a processor 1222, wherein the connector 1221 is disposed on the main body 1222 and electrically connected to the processor 1222. When the plug component 121 is assembled to the main body 122 and electrically connected to the connector 1221, the processor 1222 may analyze the physiological signals from the multi-contact connector 110 to obtain physiological information.

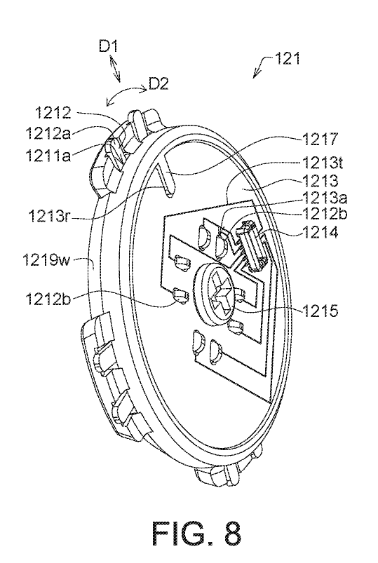

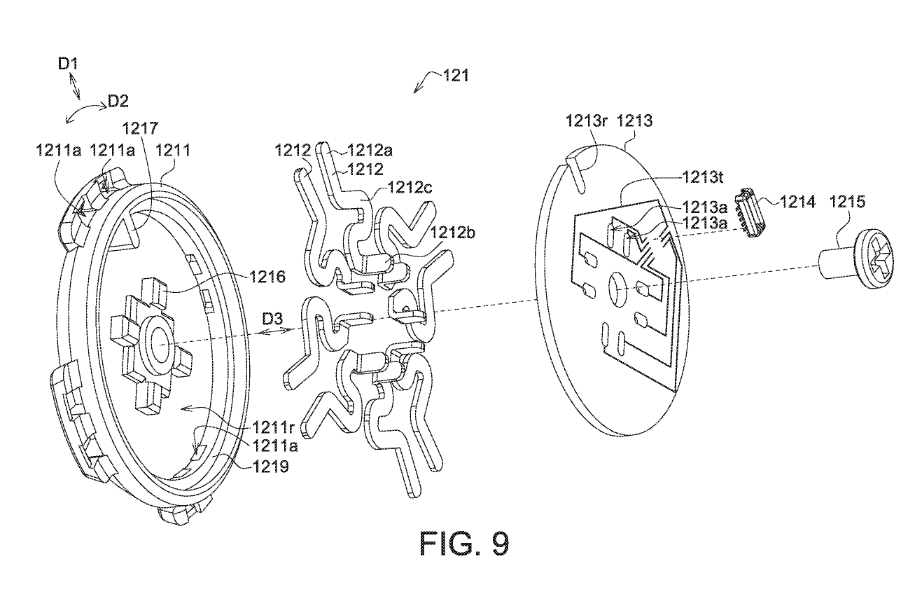

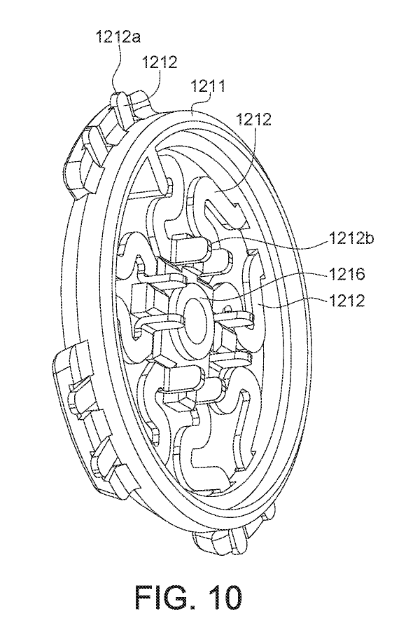

Referring to FIGS. 8 to 10, FIG. 8 shows an appearance view of the plug component 121 of FIG. 7, FIG. 9 shows an exploded view of the plug component 121 of FIG. 8, and FIG. 10 shows an assembly view of a casing 1211 and a plurality of second conductive portions 1212 of FIG. 9.

The plug component 121 includes the casing 1211, the second conductive portions 1212 (for example, eight second conductive portions 1212 in the present embodiment), a circuit board 1213, a connector 1214 and a plurality fixing elements 1215.

The fixing elements 1215s are, for example, screws or other fasteners. The fixing elements 1215 are configured to fix the second conductive portions 1212 and the circuit board 1213 to the casing 1211. In the present embodiment, the number of the second conductive portions 1212 may be equal to the number of the first conductive portions 113.

The casing 1211 includes a second receiving portion 1211r, a block 1216, a plurality of second openings 1211a (In the present embodiment, the number of the second conductive portions 1212 and the number of the second openings 1211a are eight, for example) and a protrusion 1217. The second openings 1211a pass through the circumferential wall 1219 of the casing 1211, that is, the second openings 1211a extend to an inner circumferential surface (not shown) from the outer circumferential surface 1219w of the circumferential wall 1219w for be interconnected with the second receiving portion 1211r, such that each second conductive portion 1212 of the second receiving portion 1211r located at the second receiving portion 1211r may pass through the corresponding second openings 1211a and projects from the corresponding second openings 1211, that is, projects from the outer circumferential surface 1219w of the circumferential wall 1219w. As a result, when the ECG scanner 120 and the multi-contact connector 110 connect to each other, the second conductive portions 1212 may contact the first conductive portions 113 of the multi-contact connector 110 of FIG. 2.

As shown in FIGS. 8 to 10, these second conductive portions 1212 are annularly and circumferentially disposed on the circumferential wall 1219 of the casing 1211.

As shown in FIGS. 8 and 9, each second conductive portion 1212 has a first end 1212a and a second end 1212b. The second end 1212b of each second conductive portion 1212 presses against the block 1216, and the first end 1212a of each second conductive portion 1212 passes through the corresponding second opening 1211a. The first end 1212a projects from the corresponding second opening 1211a for contacting the corresponding first conductive portions 113 of the multi-contact connector 110. In addition, the first end 1212a is substantially perpendicular to second end 1212b in an extending direction. For example, the first end 1212a extends in a radial direction D1, and the second end 1212b extends in an axial direction D3. Since the second end 1212b extends in the axial direction D3, the second end 1212b may provide a larger area for firmly pressing against the block 1216.

The first end 1212a extends in the radial direction D1, and each second conductive portion 1212 has flexibility. Furthermore, each second conductive portion 1212 further includes a flexible portion 1212c connecting the first end 1212a to the second end 1212b. The flexible portion 1212c is between and connects the first end 1212a and the second end 1212b, and the flexible portion 1212c is shaped as a bending-shape, such as U-shape, S-shape or other bending shape, for generating a normal contacting force. As a result, when the flexible portion 1212c is applied by a force, the flexible portion 1212c is deformed to provide the second end 1212b with a displacement and a normal force. In addition, each second opening 1211a has a width (in a circumferential direction D2) larger than a width (in the circumferential direction D2) of the first end 1212a, and accordingly the first end 1212a of each second conductive portion 1212 projecting from the corresponding second opening 1211a is movable in circumferential direction D2.

The circuit board 1213 has a notch 1213r and a plurality of third openings 1213 corresponding to the second conductive portions 1212. As shown in FIG. 9, the number of the third openings 1213 is eight, less than or more eight. When the circuit board 1213, the second conductive portions 1212 and the casing 1211 are assembled to each other, the second end 1212b of each second conductive portion 1212 presses against the block 1216 of the casing 1211 and passes through the corresponding third opening 1213a of the circuit board 1213. As a result, the second conductive portions 1212 may be restricted between the block 1216 and the second openings 1211a. In addition, when the circuit board 1213 is assembled to the casing 1211, the protrusion 1217 is engaged with the notch 1213r of the circuit board 1213, such that the protrusion 1217 may prevent the circuit board 1213 from rotating. As a result, the second conductive portions 1212 may be firmly fixed to between casing 1211 and the circuit board 1213.

Although not shown in FIG. 8, the plug component 121 further includes a plurality of solder (not shown), wherein each solder is connected to the second end 1212b of the corresponding second conductive portion 1212 and the corresponding trace 1213t, such that each second conductive portion 1212 is electrically connected to the corresponding trace 1213t of the circuit board 1213. The conductive wire 1213 is, for example, signal trace which extends to the connector 1214 for electrically connecting the connector 1214 to the corresponding second conductive portion 1212. As a result, the physiological signal from the second conductive portions 1212 may be transmitted to the connector 1214 through the trace 1213t.

As shown in FIGS. 7 and 8, when the plug component 121 is assembled to the main body 122, the connector 1214 of the plug component 121 may be connected to the connector 1221 of the main body 122, such that the processor 1222 disposed on the main body 122 is electrically connected to the circuit board 1213 through the connectors 1221 and 1214.

The connector 1221 is disposed on a circuit board (not shown) of the main body 122. In the present embodiment, the connector 1221 has a male port, and the connector 1214 has a female port, such that the connector 1221 and the connector 1214 may match to each other. In another embodiment, the connector 1221 has female port, and the connector 1214 has a male port, such that the connector 1221 and the connector 1214 may match to each other.

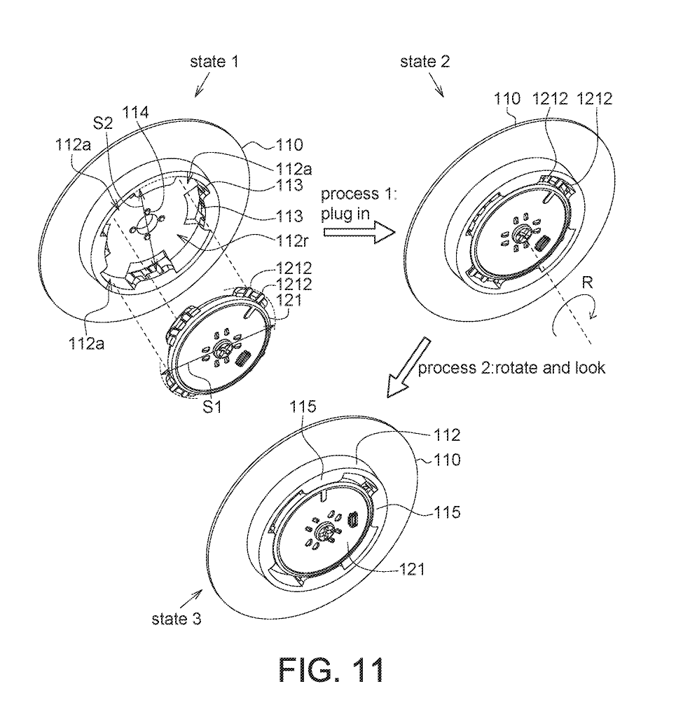

FIG. 11 shows a connecting process of the ECG scanner 120 (the main body is not shown) of FIG. 1 and the multi-contact connector 110 of FIG. 2.

In process 1 (state 1 to state 2), the plug component 121 of the ECG scanner 120 inserts the first receiving portion 112r of the multiple-contact connector 110 by way of the second conductive portions 1212 aligning with the notches 112a of the flange 112 of the multi-contact connector 110. In process 2 (state 2 to state 3), the plug component 121 is rotated around a rotating direction R, such that the plug component 121 and the multi-contact connector 110 are locked to each other. For example, use may hold the main body 122 assembled to the plug component 121 to perform the processes 1 and 2, such that the ECG scanner 120 and the multi-contact connector 110 of the cloth 10 are locked to each other. Each second conductive portion 1212 of the plug component 121 may be electrically connected to the corresponding first conductive portions 113 of the multi-contact connector 110. As a result, during once operation (process 1 to 2), the second conductive portions 1212 of the ECG scanner 120 may be electrically connected to the first conductive portions 113 of the multi-contact connector 110, such that the ECG scanner module 100 may provide a multi-contact transmitting function.

In addition, in process 2, since an outer diameter S1 formed by the outer edges of the first ends 1212a of the second conductive portions 1212 is larger than an inner diameter S2 formed by the first conductive portions 113 disposed on the inner circumferential surface (not shown) of the multi-contact connector 110, and accordingly the second conductive portions 1212 and the first conductive portions 113 are interfered with each other when the plug component 121 is rotated, and thus the second conductive portions 1212 are forced to be pressed to deform in the radial direction D1 (shown in FIG. 8). As a result, when the ECG scanner 120 and the multi-contact connector 110 connect to each other, each of the second conductive portions 1212 provides a radial pre-force to be firmly electrically connected to the corresponding first conductive portion 113. As a result, it may avoid the unsteady of the connecting impedance due to clearance fit.

In addition, in process 2, although the second conductive portions 1212 are interfered with the first conductive portions 113, due to the second conductive portions 1212 having flexibility, the second conductive portions 1212 move toward an center of the plug component 121 along the radial direction during interfering, and accordingly it may reduce the interfering resistance between the first conductive portions 113 and the second conductive portions 1212, such that the ECG scanner 120 and the multi-contact connector 110 effortlessly connect to each other by rotating.

As shown in FIG. 11, the multi-contact connector 110 further includes a plurality of first positioning portions 114. As shown in FIG. 6, the plug component 121 further includes a plurality of second positioning portions 1218. When the second conductive portions 1212 contact the first conductive portions 113 (as shown in state 3), the first positioning portions 114 are engaged with the second positioning portions 1218, and accordingly it may provide the user with an assembling feeling or an engaging feeling. Furthermore, during the relative rotation of the ECG scanner 120 and the multi-contact connector 110, if the user feels the assembling feeling or the engaging feeling, it means the second conductive portions 1212 have already contacted and electrically connected to the first conductive portions 113.

In the present embodiment, each first positioning portion 114 is, for example, a protrusion, and each second positioning portion 1218 is, for example, a dent. In another embodiment, each first positioning portion 114 is, for example, a dent, and each second positioning portion 1218 is, for example, a protrusion.

As shown in FIG. 11, the multi-contact connector 110 further includes a plurality of blocks 115 and a notch 112a formed between adjacent two blocks 115. The blocks 115 are disposed on the flange 112, extend toward an inner side of the flange 112 and at least one block 115 is opposite to the corresponding first opening 111a in position. As a result, when the second conductive portions 1212 contact the first conductive portions 113 passing through the first openings 111a (as shown in state 3), the blocks 115 are aligned with the second conductive portions 1212 of the plug component 121 in position, such that each second conductive portion 1212 may be blocked by the corresponding block 115 for preventing the plug component 121 from being easily detached from the multi-contact connector 110 in the axial direction.

In addition, each second conductive portion 1212 provides the corresponding first conductive portion 113 with the radial pre-force, that is, the second conductive portions 1212 are in contact with the first conductive portions 113 in the radial direction, and accordingly assembling of the ECG scanner 120 and the multi-contact connector 110 may save more user labour compared to the axial contact in assembling of the conventional the ECG scanner and the connector. In comparison with axial contact, the radial contact of the ECG scanner module 100 of the present embodiment may save the user labour (applying force by the user) of assembling the ECG scanner 120 to the multi-contact connector 110 by 85%. In addition, the more the number of the pairs of contacts (a pair of the contacts includes one conductive portions 1212 and the corresponding the conductive portions 113) is, the more the saved-labour is.

Moreover, as shown in formula (1) below, Fa represents the required applying force of assembling the conventional ECG scanner to the conventional multi-contact connector, n represents the number of the pairs of the contacts, and f represents the required normal force per pair of the contacts.

Fa=nf (1)

In formula (2) below, Fr represents the required applying force of assembling the ECG scanner 120 to the multi-contact connector 110, n represents the number of the pairs of the contacts, f represents the required normal force per pair of the contacts, μ represents the friction coefficient between the second conductive portions 1212 and the first conductive portions 113 which contact the second conductive portions 1212, L (as shown in FIG. 6) represents an applying-force arm, for example, a partial length of the main body 122, and r (as shown in FIG. 6) represents an applied-force arm, for example, a radius of the plug component 121, wherein r<L.

Fr=nfμ(r/L) (2)

According to the formulas (1) and (2), if r/L≅0.5 and μ≅0.3, then Fᵣ/Fₐ≅0.15. In addition, according to the formulas (1) and (2), if r/L≅0.5, n=8 and f≅60 g/contact, then Fₐ≅480 gf and Fᵣ≅72 gf.

The applying force of assembling the ECG scanner 120 to the multi-contact connector 110 is about 0.15 times the applying force of assembling the conventional ECG scanner to the conventional multi-contact connector. In addition, since the conductive portion of the ECG scanner of this invention may deform in the radial direction, the conductive portion of the ECG scanner and the conductive portion of the multi-contact connector in the present embodiment of the invention may press against each other due to the radial force resulted from the deformation in the radial direction, such that the second conductive portion may firmly press against the first conductive portion, and accordingly it may prevent the ECG scanner from being easily detached from the multi-contact connector and may increase the stability between the ECG scanner and the multi-contact connector. In comparison with the design of the conventional axial (normal) contact, the multi-contact connector has a thin thickness due to the radial contact of the conductive portion of ECG scanner and the conductive portion of the multi-contact connector. The multi-contact connector of the present embodiment of the invention may be disposed on the wearable carrier, wherein the wearable carrier is, for example, the cloth of smart clothes.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A multi-contact connector, adapted to be disposed on a wearable carrier and connected to an electrocardiography (ECG) scanner, wherein the wearable carrier has a plurality of conductive wires, the ECG scanner has a plurality of second conductive portions, each of the second conductive portions has a first end projecting from an outer circumferential surface of the ECG scanner, the multi-contact connector comprising:
a base disposed on the wearable carrier and having an inner circumferential surface;
a fitting portion disposed on the base and surrounding a first receiving portion; and
a plurality of first conductive portions annularly disposed on the inner circumferential surface, wherein each first conductive portion is electrically connected to the corresponding conductive wire;
wherein when the ECG scanner and the multi-contact connector connect to each other, each second conductive portion provides a radial force along a radial direction for being electrically connected to the corresponding first conductive portion.

2. The multi-contact connector according to claim 1, wherein the base has a plurality of first openings, the first openings pass through the base and interconnect with the first receiving portion, each first conductive portion passes through the corresponding first opening for being disposed on the inner circumferential surface of the first receiving portion, and each first conductive portion is exposed from the first receiving portion for being electrically connected to the corresponding second conductive portion.

3. The multi-contact connector according to claim 2, wherein each first conductive portion comprises a first sub-conductive portion and a second sub-conductive portion connecting to the first sub-conductive portion, each first sub-conductive portion passes through the corresponding first opening and is exposed form the first receiving portion, the first receiving portion is located at a first surface of the base, the second sub-conductive portion is disposed on a second surface of the base, and the first surface is opposite to the second surface.

4. The multi-contact connector according to claim 3, wherein the fitting portion is a flange projecting from the base, and the multi-contact connector further comprises:
at least one block disposed on the flange and extending toward an inner side of the flange, wherein the at least one block is opposite to the corresponding first opening in position.

5. The multi-contact connector according to claim 3, wherein each first sub-conductive portion and each second sub-conductive portion are shaped as plate-shape, and each first sub-conductive portion is substantially perpendicular to the corresponding second sub-conductive portion.

6. The multi-contact connector according to claim 1, wherein the wearable carrier is smart clothes.

7. An ECG scanner, adapted to be connected to a multi-contact connector having a plurality of first conductive portions, the ECG scanner comprising:
a main body; and
a plug component disposed on the main body and comprising:
a casing having a circumferential wall; and
a plurality of second conductive portions having deformability and annularly arranged in the circumferential wall of the casing, wherein each of the second conductive portions has a first end projecting from an outer circumferential surface of the circumferential wall;
wherein when the ECG scanner and the multi-contact connector connect to each other, each second conductive portion provides a radial force along a radial direction for being electrically connected to the corresponding first conductive portion.

8. The ECG scanner according to claim 7, wherein the casing further comprises a block and a second opening, the second opening passes through the circumferential wall, each of the second conductive portions has a second end pressing against the block, the first end of each second conductive portion passes through the second opening to be electrically connected to the corresponding first conductive portion.

9. The ECG scanner according to claim 8, wherein the first end and the second end of each second conductive portion are substantially perpendicular to each other in an extending direction.

10. The ECG scanner according to claim 7, wherein each second conductive portion has flexibility, an outer diameter formed by the outer edges of the first ends of the second conductive portions is larger than an inner diameter formed by the first conductive portions disposed on the inner circumferential surface of the multi-contact connector so that when the ECG scanner and the multi-contact connector connect to each other, each of the second conductive portions provides a radial pre-force to be firmly electrically connected to the corresponding first conductive portion.

11. The ECG scanner according to claim 7, wherein each second conductive portion further comprises a second end and a flexible portion, the flexible portion is between and connects the first end and the second end, and the flexible portion is shaped as a bending-shape for generating a normal contacting force.

12. An ECG scanner module, comprising:
a multi-contact connector, adapted to be disposed on a wearable carrier have a plurality of conductive wires, wherein the multi-contact connector comprises:
a base disposed on the wearable carrier and having an inner circumferential surface;
a fitting portion disposed on the base and surrounding a first receiving portion; and
a plurality of first conductive portions annularly disposed on the inner circumferential surface, wherein each first conductive portion is electrically connected to the corresponding conductive wire;
an ECG scanner configured to be connected to the multi-contact connector and comprising:
a main body; and
a plug component disposed on the main body and comprising:
a casing having a circumferential wall; and
a plurality of second conductive portions having deformability and annularly arranged in the circumferential wall of the casing, wherein each of the second conductive portions has a first end projecting from an outer circumferential surface of the circumferential wall;
wherein when the ECG scanner and the multi-contact connector connect to each other, each second conductive portion of the ECG scanner is configured to be contacted to the corresponding first conductive portion.

13. The ECG scanner module according to claim 12, wherein the base has a plurality of first openings, the first openings pass through the base and interconnect with the first receiving portion, each first conductive portion passes through the corresponding first opening for being disposed on the inner circumferential surface of the first receiving portion, and each first conductive portion is exposed from the first receiving portion for being electrically connected to the corresponding second conductive portion.

14. The ECG scanner module according to claim 12, wherein each first conductive portion comprises a first sub-conductive portion and a second sub-conductive portion connecting to the first sub-conductive portion, each first sub-conductive portion passes through the corresponding first opening and is exposed form the first receiving portion, the first receiving portion is located at a first surface of the base, the second sub-conductive portion is disposed on a second surface of the base, and the first surface is opposite to the second surface.

15. The ECG scanner module according to claim 12, wherein the multi-contact connector further comprises:
at least one block disposed on the fitting portion and extending toward an inner side of the fitting portion, wherein the at least one block is opposite to the corresponding first opening in position.

16. The ECG scanner module according to claim 14, wherein each first sub-conductive portion and each second sub-conductive portion are shaped as plate-shape, and each first sub-conductive portion is substantially perpendicular to the corresponding second sub-conductive portion.

17. The ECG scanner module according to claim 12, wherein the casing further comprises a block and a second opening, the second opening passes through the circumferential wall, each of the second conductive portions has a second end pressing against the block, the first end of each second conductive portion passes through the second opening, and the first end of each second conductive portion passing through the second opening is configured to be electrically connected to the corresponding first conductive portion.

18. The ECG scanner module according to claim 17, wherein the first end and the second end of each second conductive portion are substantially perpendicular to each other in an extending direction.

19. The ECG scanner module according to claim 12, wherein each second conductive portion has flexibility, an outer diameter formed by the outer edges of the first ends of the second conductive portions is larger than an inner diameter formed by the first conductive portions disposed on the inner circumferential surface of the multi-contact connector for, when the ECG scanner and the multi-contact connector connect to each other, each of the second conductive portions providing a radial pre-force to be firmly electrically connected to the corresponding first conductive portion.

20. The ECG scanner module according to claim 12, wherein each of the second conductive portions has a second end and a flexible portion, the flexible portion is between and connects the first end and the second end, and the flexible portion is shaped as a bending-shape.

21. A smart clothes, comprising:
the multi-contact connector as claimed in claim 1;
wherein the smart clothes covers a portion of the multi-contact connector, another portion of the multi-contact connector is exposed from the smart clothes, and the first conductive portions of the multi-contact connector are exposed from the smart clothes.

22. A smart clothes, comprising:
the ECG scanner module as claimed in claim 12;
wherein the smart clothes covers a portion of the multi-contact connector, another portion of the multi-contact connector is exposed from the smart clothes, the first conductive portions of the multi-contact connector are exposed from the smart clothes, and the ECG scanner is electrically connected to the multi-contact connector by each second conductive portion contacting the corresponding first conductive portion.
